(19) United States
(12) Patent Application Publication
(10) Pub. No.: US 2007/0050018 A1
(43) Pub. Date: Mar. 1, 2007

(54) Biodegradable stents

(76) Inventor: John Wainwright, Fremont, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/217,876

(22) Filed: Sep. 1, 2005

(51) Int. Cl. A61F 2/06

(52) U.S. Cl. 623/1.51

(57) ABSTRACT

A stent comprising a matrix and a fiber reinforcement about which the matrix is chemically or mechanically attached. The matrix is provided with heavier loads of pharmaceutically active ingredients or genetic materials as a result of the increased strength and mechanical characteristics provided to the stent by the fiber reinforcement. The fiber reinforcement can be comprised of a plurality of mono-filament fibers spaced and oriented in a flat weave pattern to which the matrix is chemically or mechanically attached. Degradation rates of the materials that comprise the matrix and the fiber reinforcement can be varied to vary the time period in which the stent maintains its mechanical characteristics or releases the pharmaceutically active ingredients or genetic materials therefrom. Multiple stage release profiles can be provided by providing multiple layers of matrices and fiber reinforcements, whereby different pharmaceutically active ingredients or genetic materials or different concentrations thereof, can be released according to the degradation profiles of the matrix and fiber reinforcment.

### FIELD OF THE INVENTION

The field of art to which this invention relates is medical devices, and in particular, stent devices made from a composite of bio-absorbable materials.

### BACKGROUND OF THE INVENTION

The use of stent medical devices, or other types of endoluminal mechanical support devices, to keep a duct, vessel or other body lumen open in the human body has developed into a primary therapy for lumen stenosis or obstruction. The use of stents in various surgical procedures has quickly become accepted as experience with stent devices accumulates, and the number of surgical procedures employing them increases as their advantages become more widely recognized. For example, it is known to use stents in body lumens in order to maintain open passageways such as the prostatic urethra, the esophagus, the biliary tract, intestines, and various coronary arteries and veins, as well as more remote cardiovascular vessels such as the femoral artery, etc. Two types of stents are generally utilized: permanent stents and temporary stents. A permanent stent is designed to be maintained in a body lumen for an indeterminate amount of time. Temporary stents are designed to be maintained in a body lumen for a limited period of time in order to maintain the patency of the body lumen, for example, after trauma to a lumen caused by a surgical procedure or by an injury, for example. Permanent stents are typically designed to provide long-term support for damaged or traumatized wall tissues of the lumen. There are numerous conventional applications for permanent stents including cardiovascular, urological, gastrointestinal, and gynecological applications. It is known that permanent stents, over time, become encapsulated and covered with endothelium tissues, for example, in cardiovascular applications. Similarly, permanent stents are known to become covered by epithelium, for example, in urethral applications.

Temporary stents, on the other hand are designed to maintain the passageway of a lumen open for a specific, limited period of time, and preferably do not become incorporated into the walls of the lumen by tissue ingrowth or encapsulation. Temporary stents may advantageously be eliminated from body lumens after a predetermined, clinically appropriate period of time, for example, after the traumatized tissues of the lumen have healed and a stent is no longer needed to maintain the patency of the lumen. For example, temporary stents can be used as substitutes for in-dwelling catheters for applications in the treatment of prostatic obstruction or other urethral stricture diseases. Another indication for temporary stents in a body lumen is after energy ablation, such as laser or thermal ablation, or irradiation of prostatic tissue, in order to control post-operative acute urinary retention or other body fluid retention.

It is known in the art to make both permanent and temporary stents from various conventional, biocompatible metals. However, several disadvantages may be associated with the use of metal stents. For example, it is known that the metal stents may become encrusted, encapsulated, epithelialized or ingrown with body tissue. Such stents are known to migrate on occasion from their initial insertion location, and are also known to cause irritation to the surrounding tissues in a lumen. Also, since metals are typically much harder and stiffer than the surrounding tissues in a lumen, this may result in an anatomical or physiological mismatch, thereby damaging tissue or eliciting unwanted biologic responses.

Further, although permanent metal stents are designed to be implanted for an indefinite period of time, it is sometimes necessary to remove permanent metal stents. For example, if there is a biological response requiring surgical intervention, often the stent must be removed through a secondary procedure. If the metal stent is a temporary stent, it will also have to be removed after a clinically appropriate period of time. Regardless of whether the metal stent is categorized as permanent or temporary, if the stent has been encapsulated, epithelialized, etc., the surgical removal of the stent will likely cause undesirable pain and discomfort to the patient and possibly additional trauma to the lumen tissue. In addition to the pain and discomfort, the patient is thus subjected to an additional time consuming and complicated surgical procedure with the attendant risks of surgery, in order to remove the metal stent.

Similar complications and problems, as in the case of metal stents, may well result when using permanent stents made from non-absorbable biocompatible polymer or polymer-composites, although these materials may offer certain benefits such as reduction in stiffness.

It is known to use bioabsorbable and biodegradable materials for manufacturing temporary stents. The conventional bioabsorbable or bioresorbable materials from which such stents are made are selected to absorb or degrade over time, thereby reducing the need for subsequent surgical procedures to remove the stent from the body lumen. In addition, it is known that bioabsorbable and biodegradable materials tend to have excellent biocompatibility characteristics, especially in comparison to most conventionally used biocompatible metals. Another advantage of stents made from bioabsorbable and biodegradable materials is that the mechanical properties can be designed to substantially eliminate or reduce the stiffness and hardness that is often associated with metal stents, which can contribute to the propensity of a stent to damage a vessel or lumen.

Bio-absorbable polymers in the context of stents have the major advantage of giving support to a body lumen while allowing the body to heal as the stent slowly dissolves, thereby minimizing the need for surgical removal of the stent, whereas metal implants or stents are either permanent or require a second procedure for removal. In addition, metal implants or stents often do not match all of the strength, modulus and toughness characteristics of the anatomical part it is replacing. Thus, while metal may work, it is often not the optimal solution.

Accordingly, there is a need for stents made from biodegradable or bio-absorbable polymers that exhibit good strength and mechanical or other characteristics akin to the body lumen in which it is emplaced, wherein the stents remain functional in a body lumen for the duration of a prescribed, clinically appropriate period of time to accomplish a predetermined therapeutic purpose, the stent thereafter degrading without breaking down into large, rigid fragments, which may cause irritation, obstruction, pain or discomfort to the patient.

### SUMMARY OF THE INVENTION

The invention relates to an implantable stent for use in body lumens. In one embodiment, the stent is comprised of a composite of bio-absorbable materials comprised of monofilament reinforcement fibers having a matrix chemically or mechanically attached around said fibers. The fibers are preferably arranged in a weaved pattern prior to the matrix being chemically or mechanically attached around said fibers. The fibers can be chemically attached to said fibers by polymerizing said matrix around said fibers. In an alternative embodiment, the matrix can be mechanically attached to said fibers by adding solubilized matrix material onto said fiber, and solidifying said solubilized matrix material on said fiber to form the composite, as by spraying the solubilized matrix material onto said fibers or dipping said fibers into said solubilized matrix material.

The matrix can be comprised of chitins, proteins, or other bio-absorbable polymers, for example, whereas the fibers can be comprised of bio-absorbable materials, such as those used for sutures, for example. Pharmaceutical ingredients, such as drugs, or other genetic materials can be loaded into the matrix prior to chemically or mechanically attaching the matrix about the fibers. The orientation and spacing of the fibers in the weaved pattern can be altered, and different fiber and matrix materials can be used to comprise the stent in order to achieve desired characteristics and degradation rates of the stent. Multiple stage release profiles for the stent can thus be achieved based on the materials used to comprise the matrix, including the drugs or other materials therein, and the fibers, and/or based on the orientation or layering of the materials used to comprise the stent. Crystallinity and strength can be imparted to the fibers by drawing or otherwise processing the monofilament fibers used to reinforce the stent. Where desired, shape memory polymers, such as PLLA or other like polymers, for example, can be used to render the stent self-expanding.

In one embodiment, the stent can be an elongate, hollow member such as a tubular structure or a helical structure. In other embodiments, the stent has a coil structure or a helical structure made from a wound fiber, and a matrix is chemically or mechanically attached to said fiber. The helical stent is made from a filament or a fiber. Optionally, the fiber is hollow. The rates of degradation of the fiber and matrix are selected such that the rate of degradation of the matrix is faster than the degradation rate of the fiber. Optionally, the fiber has in inner and outer surface, wherein the inner surface can have a different degradation rate than the outer surface.

In some cases, the matrix is designed to degrade in vivo, whereby the matrix loses it's mechanical integrity and is substantially eliminated from the lumen prior to degradation of the fibers, while the fibers remain in place. The matrix and fibers can each be made from a biodegradable polymer such as one made from ∀-hydroxy acid, chitin protein, bio-absorbable material, or combinations thereof. Non-limiting examples of biodegradable polymers include polymers made from monomers selected from the group consisting of lactide, glycolide, para-dioxanone, caprolactone, and trimethylene carbonate, caprolactone, blends thereof and copolymers thereof.

In other cases, the fibers and matrix can be made of a blend of at least two polymers or co-polymers. The blend can contain at least one faster degrading polymer and one slower degrading polymer. More specifically, the fibers can comprise a blend of at least two polymers, the first of said polymers being, for example, a glycolide-rich, lactide/glycolide copolymer containing at least 80 mole percent of polymerized glycolide, the other of said polymers being, for example, a lactide-rich copolymer containing at least 50 mole percent of polymerized lactide.

In yet other cases, the fibers comprise a blend of at least two polymers, the first of said polymers being, for example, a glycolide-rich, lactide/glycolide copolymer containing at least 80 mole percent of polymerized glycolide, and another of said polymers being, for example, a lactide-rich, lactide/glycolide copolymer, containing at least 50 mole percent of polymerized lactide.

In any case, the matrix typically degrades by hydrolysis and breaks down at a faster rate than the fibers with exposure to body fluids. The matrix releases its pharmaceutically active ingredient or genetic material and the matrix breaks down into small granular particles that are transported easily by bodily fluids. The faster degrading matrix, after sufficient in vivo exposure, possesses little or no mechanical integrity and is slowly removed, reducing the stent cross-section from a solid to a soft structure that increasingly appears to be hollow. With hydrolytic exposure, the progressively degrading stent can readily pass out of the body lumen, thereby minimizing the possibility of causing obstruction, pain or discomfort to the patient. Both the fiber and the matrix may be bio-degradable and bio-absorbable, and the pharmaceutically active ingredient or genetic material released from the matrix can be bio-absorbed, although degradation or absorption rates may differ among them. Of course, the artisan will appreciate that the fibers and the matrix can be comprised of materials that are bio-absorbable, non-bio-absorbable, or both.

Another aspect of the invention relates to a method of using stents as described herein in a surgical procedure to maintain the patency of a body lumen. The stent is inserted into the body lumen of a patient, thereby providing patency of the lumen for a specific range of times. The stent is maintained in the lumen for a sufficient period of time to effectively maintain the lumen open and to effectively let the matrix degrade and release the pharmaceutically active ingredient or other materials contained therein. Another aspect of the invention relates to a method of forming a bio-absorbable stent having the various characteristics described above, comprising providing a bio-absorbable composite material matrix; loading a pharmaceutically active ingredient or genetic material into the matrix; providing a fiber reinforcement; chemically or mechanically attaching the matrix about the fiber reinforcement; cutting the polymerized fiber reinforcement into sections; and shape-setting the sections into coiled or helical configurations.

The fiber reinforcements may be weaved into a flat pattern prior to the chemical or mechanical attachment of the matrix thereto. The degradation rates of the matrix and fibers may vary, and multiple stage release profiles may be achieved by providing layers of matrix and fiber reinforcements, whereby each layer has different degradation rates. Orienting and spacing the fibers comprising the fiber reinforcement may alter the release profile or other characteristics of the stent. At least one of the matrix and the fiber reinforcements may be comprised of shape memory polymers, thereby rendering the stent self-expanding. Such polymers may comprise at least one of PLLA or PGA. Other bio-absorbable materials used to comprise the matrix and fiber reinforcement are, for example, at least one of chitins, proteins, α-hydroxy acids, bio-degradable polymers, comprising at least one of lactice, blycolid, para-dioxanone, caprolactone, trimethylene carbonate, caprolactone and blends and co-polymers thereof.

Thus, according to the method referred to above, a stent as described herein is provided. The stent can be an elongate, hollow member and can also have a helical structure having a plurality of coils. The stent has a longitudinal axis and the coils comprising the stent have a pitch. The stent is made from a fiber having a matrix chemically or mechanically attached to said fiber. The matrix includes a pharmaceutically active ingredient or genetic material. The filament or fiber has a cross-section. The rate of degradation of the matrix can be selected to effectively provide a faster rate of degradation than the degradation rate of the fibers to effectively provide that the matrix degrades in vivo and releases the pharmaceutically active ingredients or genetic material. The matrix loses its mechanical integrity as it degrades and is substantially transported from the lumen via bodily fluids prior to degradation of the fibers. The matrix typically degrades by hydrolysis and breaks down at a faster rate than the fibers with exposure to bodily fluids. Of course, as the artisan should appreciate, the matrix can instead be comprised partially or wholly of polymeric blends that degrade slower than some or all of the fibers, which can result in the fibers degrading partially or wholly faster than the matrix.

These and other aspects of the present invention will become more apparent from the following description and examples, and accompanying drawings.

### BRIEF DESCRIPTION OF THE DRAWINGS

FIG. 1 is a perspective view of a fiber reinforcement phase weaved into a flat latticed pattern according to the systems and methods of the invention.

FIG. 2A is a cross-sectional view of a fiber reinforced stent that has been cut and shaped into a coil according to the systems and methods of the invention.

FIG. 2B is a perspective view of the stent of FIG. 2A.

FIG. 3A is a cross section view of a fiber reinforced stent that has been cut and shaped into a helical design according to the systems and methods of the invention.

FIG. 3B is a perspective view of the stent of FIG. 3A.

FIG. 4 is a perspective view of the shape of a mold for a possible fatigue/solubility screening test of polymers.

FIG. 5 is a perspective view of a cap used to align fibers for testing.

### DETAILED DESCRIPTION OF THE INVENTION

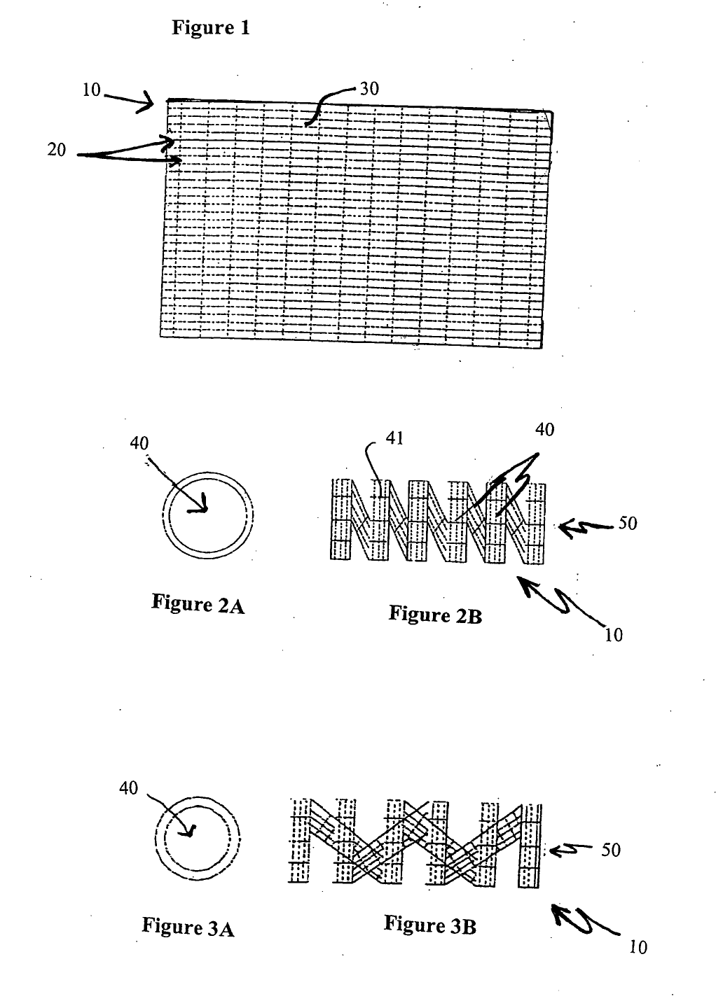

FIG. 1 illustrates materials comprising a bio-absorbable composite stent 10 according to the systems and methods of the invention. As shown in FIG. 1, stent 10 is comprised of reinforced fibers 20 and matrix 30 materials that form a bio-absorbable composite material. The fibers 20 are represented as the horizontal and vertical lines in FIG. 1, which are generally weaved into a flat lattice pattern. The matrix 30 is generally represented as the spaces between the horizontal and vertical lines, or fibers 20, of FIG. 1.

In practice, once the fibers 20 have been shaped into the flat lattice pattern and the matrix 30 has been chemically or mechanically attached thereon, strips of the composite material may be cut and shape-set into a coiled stent (FIGS. 2A & 2B) or into a helical stent (FIGS. 3A & 3B), for example. Because the matrix 30 is reinforced with the fibers 20, the matrix can be loaded with higher capacities of a pharmaceutically active ingredient, such as a drug, or other genetic material, for release from the stent 10 as the matrix 30 degrades. Such higher loading capacities in a stent can be beneficial to a patient.

The matrix 30 can be comprised of chitins, proteins, or other bio-absorbable polymers, for example, whereas the fibers can be comprised of the same of other bio-absorbable materials, such as those used for sutures, for example. As described herein, the fibers are preferably monofilament fibers to which crystallinity and strength are imparted through the drawing and processing thereof. It is preferred that fibers 20 be a continuous fiber, however, it is possible to make a stent 10 from two or more sections of fiber which are subsequently connected or hinged together. The spacing and orientation of the fibers 20 may vary from that shown in FIG. 1, as should be readily apparent to the artisan. The fibers 20, for example, may be diagonally oriented, or may be spaced further apart or closer together than shown in FIG. 1. Further although only one layer of fibers 20 is shown in FIG. 1, additional layers of fibers 20, including layers of fibers comprised of different bio-absorbable materials, may be used to comprise the stent 10 according to the systems and methods of the invention. The various layers, materials and orientations of the fibers 20 contribute to the mechanical, chemical and degradation characteristics of the stent overall, as do the materials comprising the matrices associated with any of the various layers of fibers, where provided.

In one embodiment, the stent 10 has a layer of fibers 20 and a matrix 30 chemically or mechanically attached to the fibers. For example, the matrix 30 can be coated onto the fibers 20, co-extruded with the fibers, or subsequently mounted or affixed onto fibers. The fibers can have various configurations depending upon the application including round, square, polygonal, curved, oval, and combinations thereof. Those skilled in the art will appreciate that certain cross-sectional configurations will provide different advantages or characteristics in the stent. As shown in FIGS. 2A and 2B, the stent 10 comprised of the bio-absorbable composite materials of the fibers 20 and matrix 30 can have a plurality of coils extending along a longitudinal axis 50 of the stent. The coils 40 (shown in cross-section in FIG. 2A) can be joined by hinged connecting sections 41 that help to hold the matrix 30 in place about the fibers 20. The coiled fibers thus can have a circular cross-section 40 connected together at several locations along the length of each fiber by connecting sections 41.

The fibers 20 are preferably manufactured from a bio-absorbable polymeric material as discussed above. The matrix 30 is made of bio-absorbable material as well as discussed above. Varying the materials comprising the fibers and matrix can vary the degradation rates thereof, wherein the matrix is generally comprised of a material that degrades at a rate faster than the fibers, although other degradation schedules may be provided within the scope of this invention as should be readily apparent to the artisan. The length and overall diameter of the stent will vary depending on the number of coiled sections 40 that are provided, for example. Of course, the length and diameter of the stent 10 is made to accommodate the anatomy of the patient receiving the stent and in view of the type of surgical procedure with which the stent will be deployed. Ideally the stent 10 maintains its mechanical strength and characteristics for a time duration long enough to maintain the lumen passage open and to deliver an appropriate dosing of the pharmaceutically active ingredient or other material provided in the matrix of the stent. An alternate embodiment of the stents and fibers of the present invention is to have the slower degrading polymer component as the matix and the faster degrading polymer component as the fibers. The faster degrading fibers would degrade over time leaving behind the softening matrix, which would then be transported through, or expelled or removed from, the lumen via bodily fluids, for example. The bio-absorbable polymer materials would otherwise be generally the same as for the other embodiments described herein. Pharmaceutically active ingredients or genetic material can comprise part of the matrix as described above, or may comprise part of the fibers according to the various embodiments of the systems and methods of the invention described herein.

In another embodiment, the matrix is comprised of a first biodegradable polymer composition and the fibers are comprised of a second biodegradable polymer composition. The matrix material will be selected such that the matrix will degrade by hydrolysis and lose mechanical integrity at a relatively faster rate than the fibers upon exposure to body fluids over time. The matrix material breaks down preferably into small granular particles that are easily transported or removed by bodily fluids. A portion of the fibers can be selected to have a relatively slow rate of hydrolysis that would preferably degrade or erode and expose a fibrillar morphological structure after in vivo exposure to bodily fluids. The fibrillar morphology of the fibers can aid in the dispersion of degradation products of the faster degrading matrix.

A stent is preferably designed to withstand radial stresses in order to perform the function of maintaining a passage through a lumen open. The biodegradable materials comprising the fibers of the stent according to the present invention provides the primary mechanics of withstanding radial stresses when the stent is emplaced in the body lumen. The strength, stiffness, and thickness of this material in the fibers are ideally sufficient to withstand the loads necessary to keep the stent functional and the lumen passage open. Degradation rates of the matrix material can be different than the degradation rate of the fiber material. Thus, as the matrix chemically or mechanically attached to the fibers degrades and releases pharmaceutically active ingredients or genetic material, the fibers typically have a sufficient thickness and strength to withstand loads experienced by the stent for designated time periods, before the fibers too degrade. In essence then, the fibers can be designed to fulfill the mechanical requirements of keeping the body lumen patent or open for a specified or targeted therapeutic time period.

In another embodiment, the stent is comprised of multiple layers or matrix materials to obtain a desired degradation or drug release profile. In another embodiment, the stent can employ shape memory polymers for the fibers or matrix to make the stent self expanding. Non-limiting examples of shape memory polymers include PLLA. Polymer materials useful in the stents and fibers of the present invention include those biodegradable polymers disclosed in U.S. Pat. No. 4,889,119, for example, the entire disclosure of which is hereby incorporated herein by reference. In addition, or alternatively, the matrix and fibers can each be made from a biodegradable polymer made from ∀-hydroxy acid, chitin protein, bioabsorbable material, or combinations thereof. Non-limiting examples of biodegradable polymers include polymers made from monomers selected from the group consisting of lactide, glycolide, para-dioxanone, caprolactone, and trimethylene carbonate, caprolactone, blends thereof and copolymers thereof. Ideally, the matrix comprises a polymer or polymers having a biodegradation rate faster than that of the fibers. The polymers used to manufacture the matrix can thus include polymers that hydrolyze, degrade and breakdown at a relatively faster rate compared to the material in the fibers.

When the term “caprolactone” is used herein it is meant to mean epsilon-caprolactone. These monomers can be used to make copolymers that can have random, block or segmented block sequences, or combinations thereof. Of particular utility are the segmented block copolymers of glycolide and caprolactone containing about 75 mole % of polymerized glycolide and about 25 mole % of polymerized caprolactone. Combinations of copolymers thereof can be employed.

Moreover, there can be some compatibility between the two polymers in the matrix and the fibers and the two components are somewhat immiscible. For the fibers, a blend of a glycolide copolymer containing at least 80 mole percent of polymerized glycolide can be used, the other of the said polymer being polylactide copolymer containing at least 50 mole percent of polymerized lactide.

The fibers and matrix may be formed by feeding a polymer composition to a conventional co-extruder. If desired, the fibers and matrix of the present invention can be made by other conventional processes such as melt coating, solution coating or powder coating followed by spreading the coating by melting, etc., and the like. The matrix can be added onto the fibers by polymerization, for example, as in the preferred embodiment described herein, or by melt coating or solution coating by passing the matrix through a bath, through coating rollers, and then spraying and/or die casting the matrix onto the fibers. The fibers could instead be dipped into the matrix. If it is desired to manufacture the stents of the present invention as a single tubular structure rather than a wound fiber structure, a co-extrusion process can be utilized and the co-extrusion dies would be selected to produce a tube of an appropriate diameter. Also, the fibers useful in manufacturing various embodiments of the stents, as described herein, can be manufactured to have a hollow passage through the interior or core if desired. A high-lactide polymer such as 95/5 poly(lactide-co-glycolide) can also be used to provide and retain mechanical properties over time.

Alternatively, as in U.S. Pat. No. 4,889,119, the fibers and matrix materials can be made of blends to produce absorbable plastic surgical fasteners by injection molding applications. Such materials can be used to produce fibers and matrix materials that can be made into biodegradable temporary stents.

Another variation of the stent according to the systems and methods of the invention comprises forming the fibers with a hollow fiber construction, wherein each fiber has an inner core and outer core. Each inner core and each outer core is made of materials that degrade. The materials and degradation rates of the various inner and outer cores may be the same or may differ.

In another embodiment of the stent according to the sytems and methods of the invention, the polymers and blends that form the matrix can comprise a drug delivery matrix. To form the drug delivery matrix, the polymer is mixed with a therapeutic agent, such as a pharmaceutically active ingredient or genetic material. A variety of different therapeutic agents can be used in conjunction with the polymers of the present invention. In general, therapeutic agents which may be administered via the pharmaceutically active compositions of the invention include, without limitation: anti-infectives such as antibiotics and anti-viral agents; analgesics and analgesic combinations; anti-inflammatory agents; hormones such as steroids; bone regenerating growth factors; and naturally derived or genetically engineered proteins, polysaccharides, glycoproteins, or lipoproteins.

The matrix can be formulated by mixing one or more therapeutic agents with the polymer. The therapeutic agent may be a liquid, a finely divided solid, or any other appropriate physical form. Typically, but optionally, the matrix will include one or more additives, such as diluents, carriers, excipients, stabilizers or the like.

The amount of therapeutic agent incorporated into the matrix will depend on, among other factors, the particular drug being employed and the medical condition being treated. Typically, the amount of drug represents about 0.001 percent to about 70 percent, more typically about 0.001 percent to about 50 percent, most typically about 0.001 percent to about 20 percent by weight of the matrix. The quantity and type of polymer incorporated into the drug delivery matrix will vary depending on the release profile desired and the amount of drug employed.

Upon contact with bodily fluids, the matrix polymer undergoes gradual degradation (mainly through hydrolysis) with concomitant release of the dispersed drug for a sustained or extended period. This can result in prolonged delivery (over, say 1 to 5,000 hours, preferably 2 to 800 hours) of effective amounts (say, 0.0001 mg/kg/hour to 10 mg/kg/hour) of the drug. This dosage form can be administered as necessary depending on the subject being treated, the severity of the affliction, the judgment of the prescribing physician, and the like. Following these or similar procedures, those skilled in the art will be able to prepare a variety of formulations.

In another embodiment, the fibers having a coiled or helical structure can be manufactured using a winding process. A co-extruded fiber can be used to wind the stent about a mandrel by heating the fiber and then coiling it around the mandrel. The fiber may be heated prior to winding or subsequent to winding about the mandrel using conventional processes. The assembly of the mandrel and the fibers can be annealed under constraint and then the mandrel is removed. If desired, the fibers may be annealed after removal from the mandrel. The pitch and diameter of the coils are selected to provide the desired size and shape of fibers.

Another aspect of the invention relates to a method of forming a bio-absorbable stent having the various characteristics described above, comprising providing a bio-absorbable composite material matrix; loading a pharmaceutically active ingredient or genetic material into the matrix; providing a fiber reinforcement; chemically or mechanically attaching the matrix about the fiber reinforcement; cutting the polymerized fiber reinforcement into sections; and shape-setting the sections into coiled or helical configurations.

The following examples are illustrative of the principles and practice of the present invention, although not limited thereto.

### EXAMPLE 1

A male patient, appropriately anesthetized, undergoes a prostrate thermal ablation procedure using conventional laser treatment devices. After successful completion of the surgical procedure, a stent of the present invention is inserted into the patient's urethra and bladder by methods known in the art. Prior to insertion of the stent, the surgeon trims the stent to size. The stent is placed at the end of an applicator. A conventional cystoscope is inserted into the lumen of the applicator. The stent and applicator are lubricated with a water soluble medical grade lubricant. A fluid reservoir is attached to the applicator as in standard cystoscopy procedures. The stent is placed in the prostatic urethra under direct visualization using a scope. Once positioned correctly, the applicator is removed, leaving behind the stent in the prostatic urethra. In approximately 28 days after implantation, the stent breaks down and is transported or passed from the urinary tract through normal urine voiding.

The main bio-absorbable polymers that are used in medicine are aliphatic polyesters of α-hydroxy acids and their derivatives. A non-limiting list of materials usable for comprising stents made of PLA/PGA Matrix and Fibers includes a preferred poly(α-hydroxy acids), polylactic acid, PLA, and polyglycolic acid, PGA, homopolymers and their copolymers. These polymers have a long history of use as synthetic biodegradable materials in clinical applications. Of course, the artisan will appreciate that other materials, know to the skilled artisan, are also usable with the stent comprised of a matrix and fibers having targeted degradation rates according to the systems and methods of the invention.

Measuring Degradation of Composites:

Fiber and Matrix Fabrication

One method for preparing high molecular weight PGA is ring-opening polymerization of glycolide (Reaction 1), the cyclic dimer of glycolic acid. Solution and melt polymerization methods can both be used. Typical catalysts used include organo tin, antimony, or zinc. If stannous octoate is used, temperatures of approximately 175° C. can be used for a period of 2 to 6 hours for polymerization. Similar synthesis methods could be used to make the other α-hydroxy acids. Alternatively, the α-hydroxy acids could be bought from a vendor such as Absorbable Polymers International. Polyurethanes polymerized using stannous octoate have also been used in other medical implants. Examples include cardiac pace makers using pellethane as lead insulation.

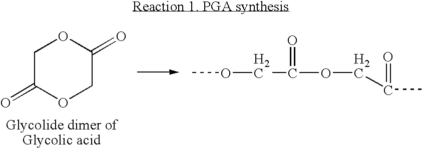

On the other hand, PLA can be prepared from the cyclic diester of lactide by the following ring opening polymerization, Reaction 2.

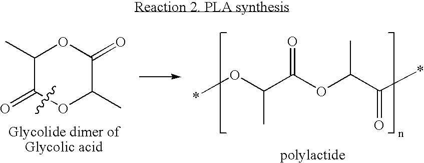

Lactic acid exists as two optical isomers or enantiomers. The L-enantiomer occurs in nature, a D,L racemic mixture results from the synthetic preparation of lactic acid. Fibers spun from “L” polylactide (mp. 170° C.) have high crystallinity when drawn whereas fibers spun from poly DL-lactide are amorphous. Crystalline poly-L-lactide are more resistant to hydrolytic degradation than amorphous DL form. Pure polyglycolide is about 50% crystalline, whereas pure poly-L-lactide is reported to be about 37% crystalline. The differences in PLA crystallinity and forms allow the initial strength and degradation profile to be tailored for the specific need. This is supported by the fact that generally, higher crystallinity of PLA-co-PGA results in higher tensile strength and

slower degradation rates in-vitro.

The different polymers shown below in Table 2 also have different melting and glass transition temperatures. This allows the compositions to be changed for the needs of the processing as well as the final Tg of the polymer.

TABLE 1
Properties of poly(∀-hydroxy acids)

| | | | Tens. | Tensile | | Mass | Strength |
|---|---|---|---|---|---|---|---|
| | Tg¹ | Tm² | Mod. | Str. | Elon- | degrad. | retention |
| Polymers | (° C.) | (° C.) | (GPa) | (Mpa) | gation | (m) | (m) |
| Poly- | 35 | 225 | 7.0 | 75 | ˜0 | 3 | 1 |
| glycolide | | | | | | | |
| (PGA) | | | | | | | |
| Poly-L- | 55 | 180 | 2.7 | 45 | 3 | 18-24 | 6-12 |
| lactide | | | | | | | |
| (PLLA) | | | | | | | |
| Poly-D,L- | 50 | None | 1.9 | n.a. | n.a. | 12-16 | 3-10 |
| lactide | | | | | | | |
| (PDLLA) | | | | | | | |
| Poly-ε- | −60 | 60 | 0.4 | 22 | >500 | >24 | >24 |
| capro- | | | | | | | |
| lactone | | | | | | | |
| (PCL) | | | | | | | |
| Poly-1,4- | −14 | 110 | 1.5 | 36 | | 4-6 | 1.5 |
| dioxane- | | | | | | | |
| 2-one | | | | | | | |
| (PDO) | | | | | | | |
| Poly- | −15 | 52 | 0.003 | 0.5 | 160 | 4-8 | n.a. |
| tri- | | | | | | | |
| methylene | | | | | | | |
| Carbonate | | | | | | | |
| (PTMC) | | | | | | | |
| Poly-β- | 5 | 175 | 2.3 | 26 | 3 | n.a. | n.a. |
| hydroxy- | | | | | | | |
| butyrate | | | | | | | |
| (PHB) | | | | | | | |

¹Glass transition temperature,
²Melting point,
³ Tensile modulus,
⁴ Tensile strength

The degree of crystallinity and mechanical properties of polymeric fibers, as used herein to comprise the fibers of the stent according to the various embodiments of the systems and methods described herein, can be greatly influenced by processing conditions. Fiber processing generally includes the following stages: an orientation stage, a hot-stretching stage and an annealing stage. The orientation roll temperature can have a significant impact on the structure formation by the process of nucleation-controlled kinetics, while the second encountered pre-annealing roll temperature is preferred for the growth of the crystallites and overall crystallinity. In the hot-stretching stage, it is found that higher hot-stretching temperature increases the tensile strength, crystallinity and crystal size. Higher hot-stretching temperature can reduce the internal stress in the restrained amorphous chains. In the annealing stage, samples can gain a significant increase in crystallinity and crystal size while heat shrinkage in the vicinity of Tg significantly decreases. The combination of the changes in PLA isomers, crystallinity and processing gives many possibilities for custom polymers.

The properties of a PLA/PGA copolymer can be tuned based on the ratios of the two polymers. A 90 mol % PGA and 10 mol % PLA ratio can be used for a biodegradable suture. A totally synthetic absorbable polymer suture made of polyglycolic acid can also be used. The icryl suture typically retains strength longer and is absorbed sooner than the PGA suture.

The viscoelastic behavior of PLA/PGA polymers makes possible a shape memory effect of the material. The stents are stable at room temperature and expansion can occur at body temperature. The expansion of a SR-PLGA A prostatic stent is the slowest. The expansion of SR-PLLA, SR-PLA 96 (96L/4D SR-PLA) and SR-PLGA C (80L/20G SR-PLGA, C=crystallized) is greatest during the first few minutes. The fastest expansion was observed in the SR-PLLA urethral stent. Of course, the artisan will appreciate that the stents can expand faster if heated, such as with the use of a heated balloon.

Accordingly, the tensile strength and the degradation of the polymer can be tuned.

If the degradation of the polymer is too fast, it may not give the body time to heal. If the degradation of the polymer is too slow, it may prevent the body from healing appropriately. Furthermore, the type of degradation is important. If surface degradation occurs, the polymer typically tends to retain its strength well into its life cycle, whereas if bulk degradation occurs, gaps in the polymer may form leading to a complete failure of the stent.

PLA/PGA has also been shown to have good biocompatibility in many different environments. For example, PLA/PGA homopolymers and co-polymers have been used to date for bone screws, rods and sutures. Moreover, in porcine coronary arteries PGA/PLA loaded stents did not have any complications at 4 weeks (van der Giessen et al. Coronary Heart Disease/Thrombosis/Myocardial-Infarction: Marked Inflammatory Sequelae to Implantation of Biodegradable and Nonbiodegradable Polymers in Porcine Coronary Arteries. Circulation, 94(7), 1690-1697 (1996.).

PLA/PGA offers many possibilities as a matrix material as well. A PLA/PGA matrix can be tuned through copolymer ratios and processing to give many different modulii and degradation profiles. In addition, the shape memory and biocompatible characteristics of a matrix give it many opportunities for use.

Polycaprolactone (PCL), which in the past has been used for sutures, for example, can be used as fiber reinforcements in the various embodiments described herein. PCL is synthesized in Reaction 3 as shown below:

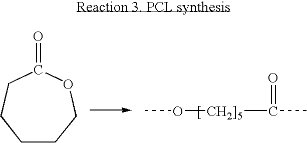

This semi-crystalline PCL polymer absorbs very slowly in vivo and releases ε-hydroxycaproic acid as a metabolite. Nonenzymatic bulk hydrolysis of ester linkages followed by fragmentation and release of oligomeric species occurs. Fragments are ultimately scavenged by macrophages and giant cells. Amorphous regions of the polymer are typically degraded prior to breakdown of the crystalline regions. PCL with an initial average molecular weight of 50,000 takes about three years for complete degradation in-vitro. The rate of hydrolysis can be altered by copolymerisation with other lactones, for example a copolymer of caprolactone and valerolactone degrades more readily. Copolymers of, -caprolactone 25% with PGA 75% can be synthesized to yield materials with more rapid degradation rates (e.g., a commercial suture MONOCRYL, Ethicon). Further, a PCL/PLA copolymer ratio can be varied. For instance, copolymers of, -caprolactone and L-lactide are elastomeric when prepared from 25% ε-caprolactone, 75% L-lactide, for example, and are rigid when prepared from 10% e-caprolactone, 90% L-lactide, for example.

Mono-filament synthetic absorbable fibers can be made from polydioxanone (PDO). The monomer p-dioxanone, is analogous to glycolide but yields a poly-(ether-ester) as shown in Reaction 4 below:

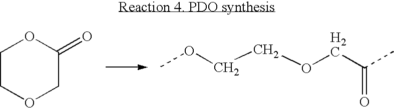

Polydioxanone monofilament fibers retain tensile strength longer than the braided polyglycolide and is absorbed in about six months with minimal tissue response. Polydioxanone degradation in vitro is affected by gamma irradiation dosage but not substantially by the presence of enzymes.

Bio-absorbable materials have been used widely clinically. The wide range of PLA/PGA copolymers allows the properties of the fibers and matrix to be tuned for the desired application. Fibers made from each of these poly(∀-hydroxy acids) can be used as the reinforcing phase. This enables the use of highly engineered materials without the added cost of processing.

Testing of Composites

Different PLLA/PGA matrix copolymers can been tested. For the PLA/PGA copolymers, resistance to hydrolysis is more pronounced at either end of the copolymers compositions range. The 70/30 PGA/PLA typically has high water uptake, hence this embodiment is readily degradable. In another embodiment, the 50/50 copolymer can be unstable with respect to hydrolysis. However, intermediate copolymers are more unstable than the homopolymers. Because of the preceding information PLLA/PGA, L/G ratio of 90/10 and 10/90 are used for the fiber and matrix.

ASTM F 1635-95 can also be used to test the composites. Six samples are taken out every seven days for tensile testing. The sample size is based on being able to detect a 2 standard deviation shift as per the following:

2-Sample t Test
Testing mean 1 = mean 2 (versus not=)
Calculating power for mean 1 = mean 2 + difference
Alpha = 0.05 Sigma = 1

| Difference | Sample Size | Target Power | | Power |
|---|---|---|---|---|
| 2 | 7 | 0.9000 | 0 | .9291 |

The mold for a test sample is made similar to the one described in ASTM F 2118-01a. The mold is longer to make room for a cap to align the fibers seen below:

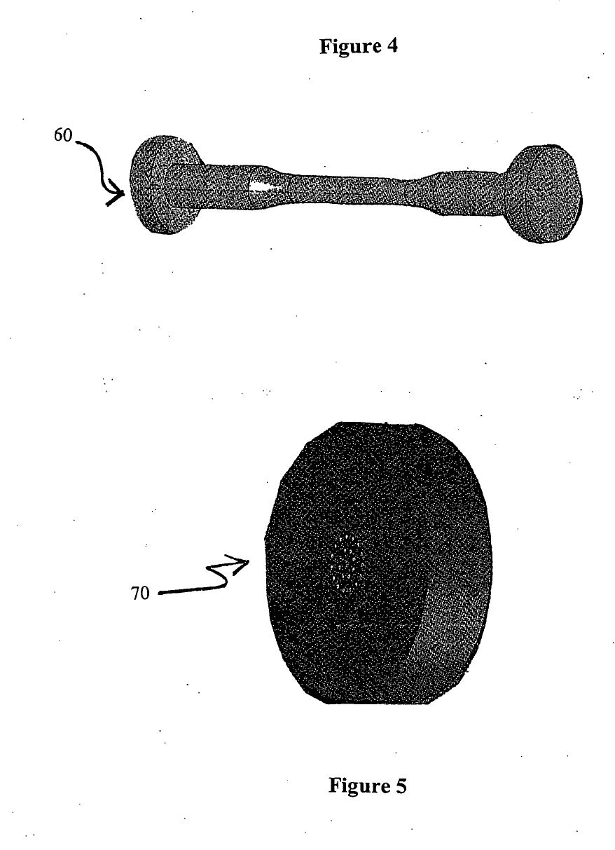

FIG. 4 (60) shows the shape of the mold for Silastic J. FIG. 5 (70) shows the cap used to align fibers.

A silicone mold is made using the shapes seen in FIGS. 4 and 5. Silastic J is used for the mold for temperatures up to 200° C. The fibers used are typically 2/0, approximately 0.014″. Therefore, 24.015″ holes are evenly spaced within a 5 mm diameter for the caps. The outer row of hole is 0.015″ from the 5 mm diameter of the test area so that no fibers are initially exposed.

1 A cap is on each side of the mold with the fibers threaded through so they are parallel to the mold length. The fibers are clamped on one end.

2 The matrix is polymerized for 1 hr using stannous octoate and a temp of 175° C. to increase the viscosity.

3 The polymer is then poured into the mold.

4 The cap is placed on the other end. The fiber is put under slight tension and clamped to fix fiber alignment during polymerization.

5 The polymerization is completed at 175° C. for 5 hrs.

6 The clamps are removed and the samples demolded.

7 The samples are then sent for EtO sterilization.

Part A: ASTM F 1635-95 are used, for example, to test the in vitro degradation of the composites. In addition, accelerated aging at 50 and 70° C. in water is tested. The accelerated aging data is used to shorten the testing time of future experiments. The composites are examined with light microscopy at 20-100× to determine mechanisms of breakdown. The samples are tensile tested to failure. The goal is to maximize the modulus after 77 days in water at body temperature. 77 days is chosen as the time it takes to reach 10ˆ7 cylcles at 1.5 Hz as described in Part B.

ASTM F 1635-95 is used, for example, to test the in vitro degradation of the composites due to hydrolysis. A mold similar to the one in ASTM F 2118-01a is used, for example. Due to the shape of the sample, most of the degradation should be of the matrix since it surrounded the fibers, but the degradation of the fibers and the interface between the fiber and matrix is seen at both ends.

In addition, accelerated aging at 50 and 70EC in water is tested. The accelerated aging data is used to shorten the testing time of future experiments. The 70EC temperature is above the Tg of both the matrix. The 50 degree temperature would be above the Tg of the 10/90 PLA/PGA matrix but below the 53EC Tg found by using the rule of mixtures of the 90/10 PLA/PGA matrix. This is used to determine if there was another degradation mechanism at temperatures above the Tg.

Part B: Selected samples in part A are then run in a DOE using a modified ASTM F 2118-01a. The cycles are run at 1.5 Hz, the temperature at 37° C. and the stress at 10 and 15 Mpa. Cylces are set to 10ˆ7 and the data plotted. The composites are examined with light microscopy at 20-100× to determine mechanism of breakdown. Any fibers that survived the 10ˆ7 cycles are tensile tested to determine the modulus.

The above methods could be modified so that the shape of the composite is closer to that of the product. Further, the drug or genetic material could be included in the matrix and the elution profile be recorded along with the physical data.

Notwithstanding the testing and material variations described herein, the various embodiments of the bio-absorbable stent described herein are comprised of a matrix and fiber reinforcement composite to accomplish a therapeutic effect over time as described herein.

Although this invention has been shown and described with respect to detailed embodiments thereof, it will be understood by those skilled in the art that various changes in form and detail may be made without departing from the spirit and scope of the claimed invention.

We claim:

1. A stent, comprising:
a matrix and a fiber reinforcement, wherein said fiber is weaved and said matrix is chemically or mechanically attached to said fiber.

2. A stent according to claim 1, wherein said matrix is chemically attached to said fiber by polymerizing said matrix around said fiber.

3. A stent according to claim 1, wherein said matrix is mechanically attached to said fiber by adding solubilized matrix material onto said fiber, and solidifying said solubilized matrix material on said fiber.

4. The stent according to claim 1, wherein said fiber is weaved into a flat pattern.

5. The stent according to claim 1, wherein said matix comprises a biodegradable polymer comprising an -hydroxy acid, chitin protein, bio-absorbable material, or combinations thereof.

6. The stent according to claim 5, wherein said biodegradable polymer comprises polymers made from monomers selected from the group consisting of lactide, glycolide, para-dioxanone, caprolactone, trimethylene carbonate, caprolactone, blends thereof and copolymers thereof.

7. The stent according to claim 5, wherein said -hydroxy acid comprises a lactide/glycolide copolymer.

8. The stent according to claim 6, wherein said lactide/glycolide copolymer has at least about 80 mole percent of polymerized glycolide.

9. The stent according to claim 6, wherein said lactide/glycolide copolymer has at least about 50 mole percent of polymerized lactide.

10. The stent according to claim 1, wherein said fiber comprises a biodegradable polymer comprising an -hydroxy acid, chitin protein, bioabsorbable material, or combinations thereof.

11. The stent according to claim 10, wherein said biodegradable polymer comprises polymers made from monomers selected from the group consisting of lactide, glycolide, para-dioxanone, caprolactone, trimethylene carbonate, caprolactone, blends thereof and copolymers thereof.

12. The stent according to claim 1, wherein the matrix is loaded with a therapeutically active agent selected from a pharmaceutically active ingredient or genetic material.

13. The stent according to claim 12, wherein the therapeutic agent is selected from anti-infectives, analgesics and analgesic combinations, anti-inflammatory agents, hormones, regenerating growth factors, and naturally derived or genetically engineered proteins, polysaccharides, glycoproteins, or lipoproteins.

14. The stent according to claim 1, wherein the matrix has a degradation rate faster than a degradation rate of the fiber.

15. The stent according to claim 1 comprising a coiled structure.

16. The stent according to claim 1 comprising a helical structure.

17. The stent according to claim 1, wherein the stent has a multiple stage release profile.

18. The stent according to claim 1, wherein multiple fibers or matrix materials can be used to obtain a desired degradation or drug release profile.

19. The stent according to claim 1, wherein shape memory polymers can be used for the fibers or matrix to make the stent self expanding.

20. The stent according to claim 19, wherein the shape memory polymer is PLLA.

21. A method of maintaining patency of a body lumen comprising inserting a stent according to claim 1 into a body lumen by a surgical procedure.

22. A method of forming a bio-absorbable stent, comprising:
Providing a bio-absorbable composite material matrix;
Loading a pharmaceutically active ingredient or genetic material into the matrix;
Providing a fiber reinforcement;
Chemically or mechanically attaching the matrix about the fiber reinforcement;
Cutting the polymerized fiber reinforcement into sections; and
Shape-setting the sections into coiled or helical configurations.

23. The method according to claim 22, wherein providing the bio-absorbable composite material matrix further comprises providing the matrix having a degradation rate faster than a degradation rate of the fiber reinforcement.

24. The method of claim 22 further comprising providing multiple stage release profiles by providing layers thereof the matrix and the fiber reinforcement, each layer being comprised of bio-absorbable materials having different degradation rates.

25. The method of claim 23, wherein providing the fiber reinforcement comprises providing a plurality of fibers.

26. The method of claim 25, wherein each fiber is hollow, having an outer core and an inner core, the outer core having a different degradation rate than the inner core.

27. The method of claim 26, wherein the degradation rate of the outer core is faster than the degradation rate of the inner core.

28. The method according to claim 26, further comprising orienting and spacing the mono-filament fibers of the fiber reinforcement to alter characteristics of the stent.

29. The method according to claim 26, further comprising providing at least one of the matrix and the fiber reinforcement with shape memory polymers to render the stent self-expanding.

30. The method of claim 23, wherein the polymer is at least one of PLLA or PGA.

31. The method of claim 22, wherein the matrix and the fiber reinforcement is comprised of bio-absorbable materials comprising at least one of chitins, proteins, -hydroxy acids or other bio-absorbable material, or of bio-degradable polymers comprising at least one of lactice, blycolid, para-dioxanone, caprolactone, trimethylene carbonate, caprolactone and blends and co-polymers thereof.

32. The method of claim 22, wherein the matrix and fiber reinforcement is comprised of a blend of at least two polymers or co-polymers, one of which degrades faster than the other.

33. The method of claim 32, wherein one polymer or co-polymer contains at least 80 mole percent polymerized glycolide and the other polymer or co-polymer contains at least 50 mole percent polymerized lactide.
